(19) United States
(12) Patent Application Publication
(10) Pub. No.: US 2020/0114026 A1
(43) Pub. Date: Apr. 16, 2020

(54) Lighting System for an Enclosed Space

(71) Applicant: Hubbell Incorporated, Shelton, CT (US)

(72) Inventors: Bruce Robeson Rhodes, Inverness, IL (US); Joseph P. Kunst, Lockport, IL (US); John DiNardi, Palos Park, IL (US); Blake Ashton Nickles, Greenville, SC (US); Anthony Sinphay, Greenville, SC (US); Brandon Cartwright, San Jose, CA (US)

(21) Appl. No.: 16/600,653

(22) Filed: Oct. 14, 2019

Related U.S. Application Data
(60) Provisional application No. 62/745,725, filed on Oct. 15, 2018.

(51) Int. Cl. A61L 2/08; F21V 23/04; H05B 47/16

(52) U.S. Cl. CPC A61L 2/084; H05B 47/16; F21V 23/0457

(57) ABSTRACT

A lighting system for an enclosed space associated with a non-occupancy function. The lighting system can include one or more HINS light sources disposed within the enclosed space associated with the non-occupancy function. The one or more light sources can be configured to emit HINS light onto one or more surfaces defining the enclosed space.

### PRIORITY CLAIM

The present application claims the benefit of priority of U.S. Provisional App. No. 62/745,725, titled “Lighting System for an Enclosed Space,” having a filing date of Oct. 15, 2018, which is incorporated herein by reference.

### FIELD

The present subject matter relates generally to lighting systems.

### BACKGROUND

Mold and bacteria can grow on surfaces defining an enclosed space found in, for example, an appliance. As such, the surfaces must be cleaned periodically to prevent mold and bacterial growth. The surfaces can be manually cleaned by a person. However, manually cleaning the surfaces can be labor intensive. Lighting systems configured to emit ultraviolet light onto the surfaces can supplement or replace the need to manually clean the surfaces. However, the ultraviolet light can be harmful to the surfaces.

### SUMMARY

Aspects and advantages of embodiments of the present disclosure will be set forth in part in the following description, or may be learned from the description, or may be learned through practice of the embodiments.

One example aspect of the present disclosure is directed to a lighting system for an enclosed space associated with a non-occupancy function. The lighting system can include one or more HINS light sources disposed within the enclosed space associated with the non-occupancy function. The one or more light sources can be configured to emit HINS light onto one or more surfaces defining the enclosed space.

Another example aspect of the present disclosure is directed to a method for controlling a lighting system for an enclosed space associated with storing inanimate objects. The method can include obtaining, by one or more control devices, data indicative of a trigger event. The method can further include activating, by the one or more control devices, one or more high intensity narrow spectrum (HINS) light sources of the lighting system in response to obtaining the data indicative of the trigger event. The one or more HINS light sources can be disposed within the enclosed space associated with storing inanimate objects such that the one or more or more HINS light sources emit light onto one or more surfaces defining the enclosed space.

Yet another example aspect of the present disclosure is directed to a lighting system for an enclosed space associated with an appliance. The lighting system can include one or more HINS light sources disposed within the enclosed space associated with the appliance. The one or more light sources can be configured to emit HINS light onto one or more surfaces defining the enclosed space.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 depicts components of a lighting system according to example embodiments of the present disclosure;

FIG. 2 depicts a block diagram of a control device of a lighting system according to example embodiments of the present disclosure;

FIG. 3 depicts components of the lighting system of FIG. 1 disposed within an enclosed space according to example embodiments of the present disclosure;

FIG. 4 depicts a door defining a portion of an enclosed space in a closed position according to example embodiments of the present disclosure;

FIG. 5 depicts a door defining a portion of an enclosed space in an open position according to example embodiments of the present disclosure; and

FIG. 6 depicts a flow diagram of an example method for controlling operation of a lighting system for an enclosed space according to example embodiments of the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Example aspects of the present disclosure are directed to a lighting system. The lighting system can include one or more high intensity narrow spectrum (HINS) light sources disposed within an enclosed space associated with a non-occupancy function. As used herein, an enclosed space associated with a non-occupancy function refers to a space that is substantially enclosed and is not intended to be occupied by persons or animals. An enclosed space associated with a non-occupancy function can be a space intended to store inanimate objects (e.g., food).

In some implementations, the enclosed space can be associated with an appliance (e.g., ice maker, refrigerator, washing machine, dishwasher, freezer, etc.). The HINS light source(s) can be used to illuminate one or more surfaces defining the enclosed space for antimicrobial purposes. In this manner, the HINS light can reduce, eliminate, suppress and/or inactivate bacterial, fungal, viral, and/or other microorganism contamination on such surfaces.

In some implementations, the enclosed space can be defined, at least in part, by a door movable between an open position and a closed position to permit selective access to the enclosed space. The HINS light sources can be operated based, at least in part, on a position of the door. For instance, when the door is in the closed position, the HINS light sources can be activated to illuminate one or more surfaces defining the enclosed space with HINS light. In some implementations, the HINS light sources illuminate the surfaces until the door moves to the open position. In alternative implementations, the HINS light sources can illuminate the surfaces with HINS light for a predetermined amount of time needed to eliminate the presence of microorganisms on the surfaces.

In some implementations, the lighting system can include one or more non-HINS light sources disposed within the enclosed space. The one or more non-HINS light sources can be configured to emit non-HINS light when the door is in the open position. For instance, the one or more non-HINS light sources can be configured to emit light having a color temperature associated with white light. In some implementations, both the HINS light sources and the non-HINS light sources can illuminate the surfaces when the door is moving from the closed position to or towards the open position. In this manner, the surfaces can be illuminated with a blend of light that includes both HINS light and non-HINS light.

In some implementations, the lighting system can include an optical sensor (e.g., spectrometer) disposed within the enclosed space and configured to measure one or more optical properties (e.g., wavelength) of light emitted by the HINS light sources. The lighting system can further include one or more output devices external to the enclosed space. The output devices can be configured to provide one or more indicators indicative of operation of the HINS light sources. In some implementations, the one or more indicators can indicate the HINS light sources are emitting HINS light. Alternatively or additionally, the one or more indicators can indicate the HINS light sources are not emitting HINS light.

In some implementations, the one or more indicators can be indicative of a wavelength shift by the HINS light sources. The wavelength shift can indicate the HINS light sources no longer emit light in the HINS wavelength (e.g., about 380 nanometers (nm) to about 420 nm, such as about 400 nm to 420 nm). In this manner, the one or more indicators can indicate the HINS light sources need to be replaced.

In some implementations, the lighting system can be configured to communicate the one or more indicators can be communicated as one or more data packets or other data transmission over a network (e.g., a wired network, a wireless network, or a combination thereof) to a computing system. The computing system can be any suitable computing device or system, such as one or more laptops, desktops, servers, smartphones, wearable devices (e.g., smart watches), tablets, or other computing devices. The computing system can process the data packets or other data transmission and provide a notification to users, premise occupants, technicians, and/or others through a suitable user interface and/or output device. The computing system can store the one or more data packets or other data transmission in a suitable memory device so as to retain historical information indicative of operation of the lighting system, specifically the HINS light sources.

As used herein, a lighting system can include, but is not limited to, one or more of a lighting circuit, light engine, one or more luminaires, one or more lighting units, a plurality of lighting devices arranged within an enclosed space, or a combination of any of the foregoing. HINS light refers to light having at least one peak wavelength in the range of about 380 nanometers (nm) to about 420 nm, such as about 400 nm to about 420 nm, such as about 405 nm. Non-HINS light refers to light in the visible spectrum, but not in the HINS range of 380 nm to 405 nm. As used herein, use of the term “about” in conjunction with a numerical value refers to within 5% of the stated numerical value.

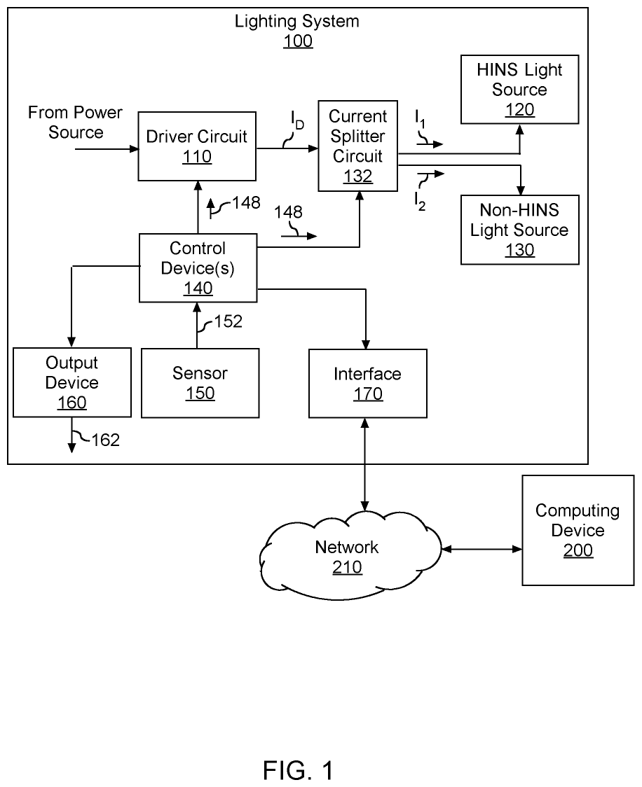

Referring now to the figures, FIG. 1 depicts a block diagram of components of a lighting system 100 according to example embodiments of the present disclosure. As shown, the lighting system 100 can include a driver circuit 110 configured to receive input power (e.g., AC or DC power) from a power source and convert the input power to a driver output (e.g., driver current) I_{D} suitable for powering one or more HINS light sources 120 of the lighting system 100. The driver output I_{D} can be provided to the one or more HINS light sources 120 over a suitable channel (e.g., one or more conductors, circuit paths, traces, etc.). In some implementations, the lighting system 100 can include non-HINS light sources 130. In such implementations, the driver circuit 110 can be configured to provide the driver current I_{D} to the one or more non-HINS light sources 130 over a suitable channel (e.g., one or more conductors, circuit paths, traces, etc.).

In some implementations, the HINS light sources 120 can include one or more HINS light emitting diode (LED) devices (e.g., a HINS LED array) configured to emit HINS light. The HINS LEDs can include one or more LED devices configured to emit HINS light as a result of electrons moving through a semiconductor material. In some implementations, the LED devices can include one or more coating, lenses, material, etc. that transform light emitted by the LED devices into HINS light. Although the HINS light sources 120 are discussed with reference to LED devices, it should be appreciated that other types of light sources configured to emit HINS light can be used without deviating from the scope of the present disclosure.

In some implementations, the non-HINS light sources 130 can include one or more non-HINS LED devices (e.g., a non-HINS LED array) configured to emit non-HINS light. The non-HINS LED can include one or more LED devices configured to emit non-HINS light as a result of electrons moving through a semiconductor material. The LED devices can be configured to emit non-HINS light of any suitable color/and or temperature. Although the non-HINS light sources 130 are discussed with reference to LED devices, it should be appreciated that other types of light sources configured to emit HINS light can be used without deviating from the scope of the present disclosure.

The driver circuit 110 can be any suitable driver circuit configured to convert an input power (e.g., an input AC or DC power) to a suitable driver output (e.g., driver current) for powering the HINS light sources 120 and/or the non-HINS light sources 130. In some implementations, the driver circuit can be a dimmable driver circuit. The driver circuit can be a multi-channel driver circuit configured to power the HINS light sources 120 over a first channel and the non-HINS light sources 130 over a second channel. Other suitable arrangements can be used to provide power to the HINS light sources and/or non-HINS light sources without deviating from the scope of the present disclosure. For instance, independent driver circuits can be used to power the HINS light sources 120 and the non-HINS light sources 130. In some implementations, the lighting system 100 can include a current splitter circuit 132 configured to allocate the driver current I_{D} between the HINS light sources 120 and the non-HINS light sources 130. More specifically, the current splitter circuit 132 can be configured to split the driver current I_{D} into a first current I₁ and a second current I₂. The first current I₁ can be provided to the HINS light sources 120. The second current I₂ can be provided to the non-HINS light sources 130.

The driver circuit 110 can include various components, such as switching elements (e.g., transistors) that are controlled to provide the driver output I_{D} for the HINS light sources 120 and/or non-HINS light sources 130. For instance, in some implementations, the driver circuit 110 can include one or more transistors. Gate timing commands can be provided to the one or more transistors to convert the input power to the driver output I_{D} using pulse width modulation techniques. In some implementations, the driver circuit 110 can be a line dimming driver, such as a phase-cut dimmable driver, Triac dimmer, trailing edge dimmer, or other line dimming driver.

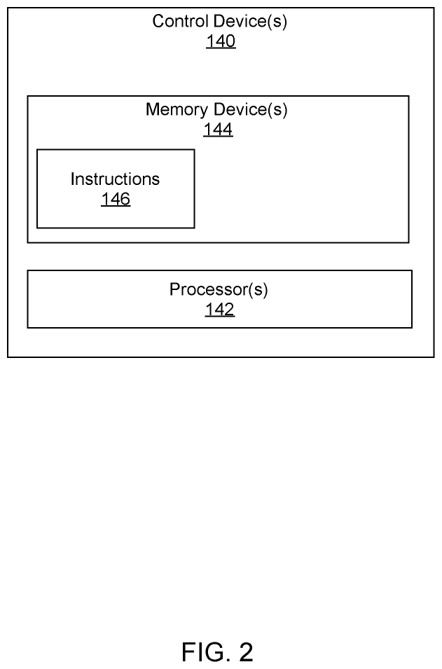

In some implementations, the lighting system 100 can include one or more control devices 140. FIG. 2 illustrates one embodiment of suitable components of the control devices 140. As shown, the control devices 140 can include one or more processors 142 configured to perform a variety of computer-implemented functions (e.g., performing the methods, steps, calculations and the like disclosed herein). As used herein, the term “processor” refers not only to integrated circuits referred to in the art as being included in a computer, but also refers to a controller, microcontroller, a microcomputer, a programmable logic controller (PLC), an application specific integrated circuit (ASIC), a Field Programmable Gate Array (FPGA), and other programmable circuits.

As shown, the control devices 140 can include one or more memory devices 144. Examples of the memory devices 144 can include computer-readable media including, but not limited to, non-transitory computer-readable media, such as RAM, ROM, hard drives, flash drives, or other suitable memory devices. The memory devices 144 can store information accessible by the processors 142, including computer-readable instructions 146 that can be executed by the processors 142. The computer-readable instructions 146 can be any set of instructions that, when executed by the processors 142, cause the processors 142 to perform operations. The computer-readable instructions can be software written in any suitable programming language or can be implemented in hardware.

In some implementations, the computer-readable instructions 146 can be executed by the control devices 140 to perform operations, such as generating one or more control actions to control operation (e.g., activate and deactivate) the HINS light sources 120 (FIG. 2) and/or the non-HINS light sources 130. For instance, the control actions can include controlling the driver output I_{D} (FIG. 2) provided to the HINS light sources 120 and/or the non-HINS light sources 130. In this manner, the control devices 140 can control the lumen output of the HINS light sources 120 and/or the non-HINS light sources.

In some implementations, the control devices 140 can provide a control signal 148 to the driver circuit 110 to control the driver output I_{D} and illumination of the HINS light sources 120 and/or the non-HINS light sources 130. Alternatively, the control devices 140 can be configured to provide the control signal 148 to the current splitter circuit 132 to control the ratio between the first current I₁ and the second current I₂ the current splitter circuit 132 provides to the HINS light sources 120 and non-HINS light sources 130, respectively. The control signal 148 can be any suitable control signal, such as a 0 Volt (V) to 10 V control signal, a DMX control signal, or a DALI control signal. It should be appreciated that the control signal can be communicated over any suitable wired, wireless, or combination of wired and wireless communication media using any suitable protocol.

Example communication technologies can include, for instance, Bluetooth low energy, Bluetooth mesh networking, near-field communication, Transport Layer Security (TLS), Wi-Fi (e.g., IEEE, 802.11), Wi-Fi Direct (for peer-to-peer communication), Z-Wave, Zigbee, Halow, cellular communication, LTE, low-power wide area networking, VSAT, Ethernet, MoCA (Multimedia over Coax Alliance) Power-line communication (PLC), digital line transmission (DLC), etc. Other suitable wired and/or wireless communication technologies can be used without deviating from the scope of the present disclosure.

Referring again to FIG. 1, the lighting system 100 can include an optical sensor 150. The optical sensor 150 can include, for instance, a spectrometer, one or more photodiodes, devices for converting light into electrical signals, other sensors, or any combination of the foregoing. It should be appreciated that the optical sensor 150 can be arranged to be in a path of the HINS light emitted by the HINS light sources 120. The optical sensor can generate one or more signals 152 associated with characteristics of the light emitted by the HINS light sources 120, such as signals associated with the wavelength(s) of light emitted by the HINS light sources 120 (e.g., peak wavelengths) and/or signals associated with the intensity of light emitted by the HINS light sources 120. It should be appreciated that the optical sensor 150 can be communicatively coupled to the control devices 140 via any suitable wired or wireless communication link. In this manner, the signals 152 generated by the optical sensor 150 can be provided to the control devices 140.

It should also be appreciated that other suitable sensors can be used in the lighting system 100 without deviating from the scope of the present disclosure. For example, the temperature sensors or other sensors configured to monitor operational performance and/or characteristics of the HINS light emitted by the HINS light sources 120. For instance, in some embodiments, one or more current sensors can be configured to detect the driver current I_{D} provided to the HINS light sources 120. In this manner, the one or more current sensors can detect operation of the HINS light sources 120 based, at least in part, on the driver current I_{D}.

In some implementations, the lighting system 100 can include an output device 160 configured to provide an indicator 162 (e.g., visual, audio, electronic data type) indicative of operation of the lighting system 100. For example, the output device 160 can include a display screen (e.g., LED screen) configured to display a visual indicator. Alternatively or additionally, the output device 160 can include one or more speakers or other audio output devices configured to provide an audio indicator, such as a beep, tone, sound, voice, speech, or other suitable type of audio indicator.

In some implementations, the lighting system 100 can provide multiple indicators 162 with each indicator being indicative of different characteristics or parameters of the HINS light sources 120 and/or HINS light. For instance, the output device 160 can provide a first indicator when the HINS light sources 120 are providing HINS light at a level sufficient for antimicrobial purposes. The output device 160 can provide a second indicator when the HINS light sources 120 are no longer emitting HINS light (e.g., due to a wavelength shift). The first indicator can be different from the second indicator. For example, the first indicator can be a first visual signal (e.g., a green illuminated LED) provided by the output device 160. The second indicator can be a second visual signal (e.g., a red illuminated LED) provided by the output device 160. As another example, the first indicator can be an audio signal provided by the output device 160. In addition, the second indicator can be the second visual signal. As yet another example, the first indicator can be a first electronic data type and the second indicator can be a second electronic data type. Various different combinations of indicators can be used to provide information concerning operation and performance of the HINS light sources 120 and/or parameters of the HINS light without deviating from the scope of the present disclosure.

In some implementations, the driver circuit 110, HINS light sources 120, non-HINS light sources 130, and control devices 140 can be implemented on the same circuit board. The optical sensor 150 and output device 160 can be located remote from the driver circuit 110, HINS light sources 120, non-HINS light sources 130, and control devices 140. In some implementations, the control devices 140 can control the output device 160 over a suitable communication medium, such as a wired or wireless communication medium.

In some implementations, the control devices 140 can be configured to provide an indicator as electronic data. The electronic data can be provided in any suitable format and/or protocol. The electronic data can be indicative of operational performance and/or characteristics of the HINS light sources 120. For example, the electronic data can indicate that HINS light is no longer being emitted by the HINS light sources 120 (e.g., as a result of wavelength shift by the HINS LEDs). In some embodiments, the electronic data can indicate that the HINS light is being provided at a level sufficient for dosing purposes (e.g., for antimicrobial purposes). The electronic data can include other information, such as time stamps and other parameters.

In some implementations, the control devices 140 can be coupled to an interface 170 for communicating electronic data and other signals to a remote location. The interface 170 can include one or more components for communication electronic data to a remote device, for instance, over suitable communication media. For example, the interface 170 can include one or more circuits, terminals, pins, contacts, conductors, transmitters, transceivers, ports, or other components for communicating electronic data to a remote location.

In some implementations, the control devices 140 can be configured to communicate indicators 162 as electronic data to remote devices, such as remoting computing devices 200 over a network 210. The network 210 can include, for instance, one or more of a local area network, a home area network, a cellular network, a wide area network, the Internet, or other suitable network. The network 210 can include any combination of wired and/or wireless communication media and can be configured to communicate data using any suitable protocol.

In some implementations, the control devices 140 can communicate electronic data as data packets or other data transmission over the network 210 to remote computing devices, such as remote computing device 200. Computing device 200 can be any suitable computing device (e.g., laptop, desktop, server, web server, smartphone, tablet, wearable devices, etc.). The computing device 200 can include one or more processors and one or more memory devices. The processors can be configured to execute computer-readable instructions stored in the one or more memory devices to process and store the electronic data received from the lighting system 100, specifically the control devices 140.

In some implementations, the indicators received as electronic data at the computing device 200 can be presented to a user through a suitable user interface or through an output device (e.g., as visual or audio notifications). The electronic data can also be stored as data (e.g., in one or more databases) in the one or more memory devices to allow users to access historical information concerning operational performance and/or characteristics of the HINS light sources 120, parameters of the HINS light, and/or other components of the lighting system 100.

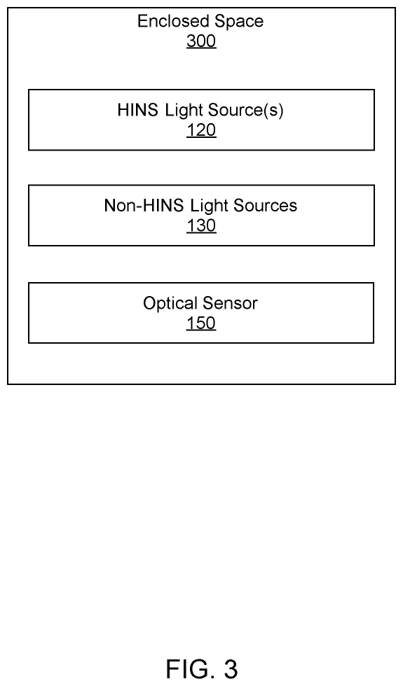

Referring now to FIG. 3, the HINS light sources 120 and the optical sensor 150 of the lighting system 100 can be disposed within an enclosed space 300 associated with a non-occupancy function. In some implementations, the enclosed space 300 can be associated with an appliance, such as a refrigerator or icemaker. For instance, the enclosed space 300 can be configured to accommodate inanimate objects (e.g., ice cubes, food items, canned beverages, etc.). Additionally, the one or more non-HINS light sources 130 can be disposed within the enclosed space 300. In example embodiments, the enclosed space 300 can be associated with an appliance, such as an icemaker, refrigerator, washing machine, dishwasher, or any other suitable appliance defining an enclosed space 300.

In some implementations, the HINS light sources 120 can be configured to illuminate one or more surfaces defining the enclosed space 300. The optical sensor 150 can be positioned on the one or more surfaces illuminated by the HINS light sources 120. As discussed above, the optical sensor 150 can generate signals 152 (FIG. 1) associated with characteristics of the light emitted by the HINS light sources 120, such as signals associated with the wavelength(s) of light emitted by the HINS light sources 120 (e.g., peak wavelengths) and/or signals associated with the intensity of light emitted by the HINS light sources 120. It should be appreciated that the optical sensor 150 can be communicatively coupled to the control devices 140 (FIG. 1) via any suitable wired or wireless communication link. In this manner, the signals 152 generated by the optical sensor 150 can be provided to the control devices 140. It should be appreciated that the output device 160 (FIG. 1) of the lighting system 100 can be external to the enclosed space 300. In this manner, one or more users can see or hear the indicator 162 (FIG. 1) indicative of operation and performance of the HINS light sources 120 and/or parameters of the HINS light.

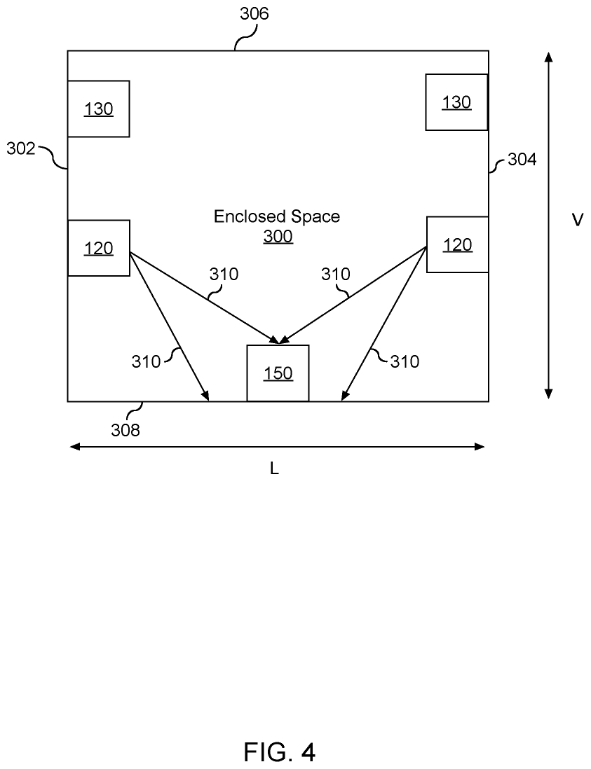

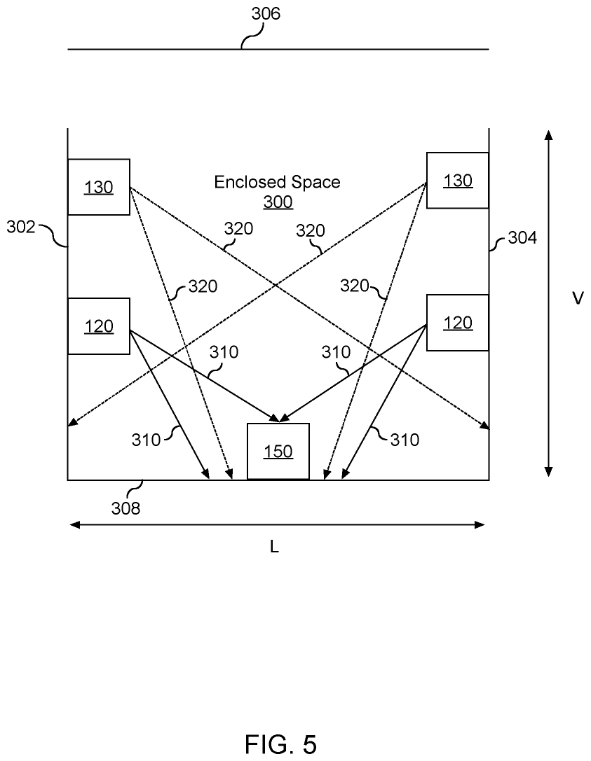

Referring now to FIGS. 4 and 5, the enclosed space 300 can be defined by a first side 302 and a second side 304 spaced apart from the first side 302 along a lateral direction L. Additionally, the enclosed space 300 can be further defined by a top 306 and a bottom 308 spaced apart from the top 306 along a vertical direction V. As shown, HINS light sources 120 can be positioned along the first side 302 and the second side 304. Additionally, non-HINS light sources 130 can be positioned along the first side 302 and the second side 304. In some implementations, the HINS light sources 120 and/or the non-HINS light sources 130 can be secured to the first side 302 and the second side 304 via a track configured to allow the HINS light sources 120 and/or the non-HINS light sources 130 to move along the vertical direction V. For instance, the control devices 140 (FIG. 1) can be configured to control operation of a motor (not shown) to move the HINS light sources 120 and/or non-HINS light sources 130 along the track.

In alternative implementations, the control devices 140 can be configured to control operation of one or more electric motors to rotate (e.g., tilt) the HINS light sources 120 and/or non-HINS light sources 130 up or down along the vertical direction V. Alternatively or additionally, the control devices 140 can be configured to control operation of the electric motors to rotate (e.g., tilt) the HINS light sources 120 and/or non-HINS light sources 130 from side to side along a longitudinal direction (not shown) that is orthogonal to both the vertical direction V and lateral direction L. It should be appreciated, however, that the control devices 140 can be configured to rotate the HINS light sources 120 and/or non-HINS light sources 130 along any suitable axis to increase a surface area (e.g., interior surfaces of enclosed space 300) illuminated by the HINS light sources and/or non-HINS light sources.

In some implementations, the top 306 can be a door or cover movable between a closed position (FIG. 4) and an open position (FIG. 5) to permit selective access to the enclosed space 300. The control devices 140 (FIG. 1) can obtain data from one or more sensors (not shown) configured to detect a position of the door. When the data indicates the door is in the closed position (FIG. 4), the control devices 140 can activate the HINS light sources 120. When the HINS light sources 120 are activated, HINS light 310 can illuminate one or more surfaces defining the enclosed space 300. For instance, the HINS light 310 can illuminate an interior surface of the bottom 308, an interior surface of the first side 302, and an interior surface of the second side 304. In some embodiments, the interior surfaces can be reflective. In this manner, the HINS light 310 can reflect off of one interior surface and onto another interior surface, such as the interior surface of the door (e.g., top 306).

When the data indicates the door is in the open position (FIG. 5), the control devices 140 can activate the non-HINS light sources 130. When the non-HINS light sources 130 are activated, non-HINS light 320 can illuminate one or more surfaces defining the enclosed space 300. For instance, the non-HINS light 320 can illuminate an interior surface of the bottom 308, an interior surface of the first side 302, and an interior surface of the second side 304. In some embodiments, the HINS light 310 and the non-HINS light can blend with one another to create light having a color characteristic similar to white light.

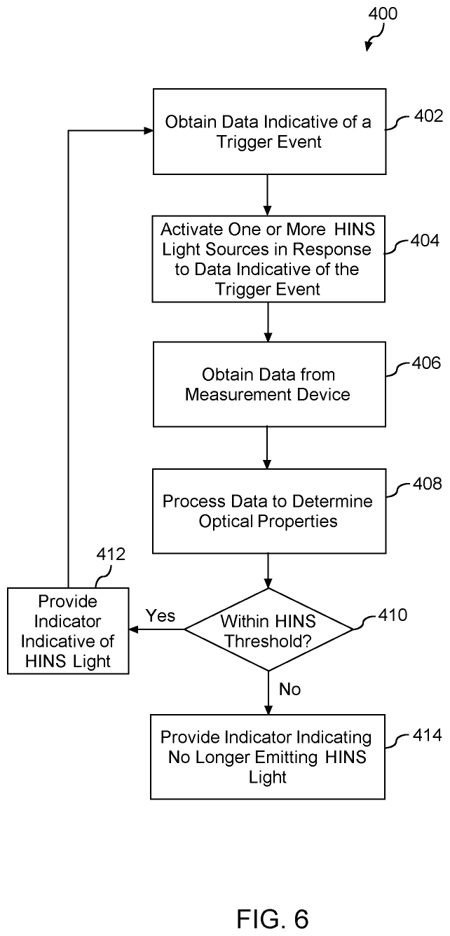

Referring now to FIG. 6, a flow diagram of a method 400 for controlling operation a lighting system for an enclosed space associated with storing inanimate objects (e.g., food) according to example embodiments of the present disclosure. In general, the method 400 will be discussed with reference to the lighting system 100 described above with reference to FIGS. 1 through 5. However, it should be appreciated by those of ordinary skill in the art that the disclosed method 400 can generally be implemented with any suitable enclosed space. In addition, although FIG. 6 depicts steps performed in a particular order for purposes of illustration and discussion, the method 400 discussed herein is not limited to any particular order or arrangement. One skilled in the art, using the disclosure provided herein, will appreciate that various steps of the method 400 disclosed herein can be omitted, rearranged, combined, and/or adapted in various ways without deviating from the scope of the present disclosure.

At (402), the method 400 includes obtaining, by one or more control devices, data indicative of a trigger event. In some implementations, the trigger event can be associated with movement of a door defining a portion of the enclosed space. For instance, the data can indicate movement of the door from an open position to a closed position. In alternative implementations, the one or more control devices can be configured to activate the HINS light sources at predetermined intervals of time. In such implementations, the trigger event can comprise passage of a predetermine amount of time since the HINS light sources were last activated. In response to obtaining data indicative of the trigger event, the method 400 proceeds to (404).

At (404), the method 400 includes activating, by the one or more control devices, one or more HINS light sources disposed within the enclosed space. In this manner, one or more interior surfaces defining the enclosed space can be illuminated with HINS light for antimicrobial purposes. In some implementations, the HINS light sources can be activated until the one or more control devices obtain data indicative of the door moving from the closed position to or towards the open position. In alternative implementations, the HINS light sources can be activated for a predetermined amount of time corresponding to an amount of time needed to reduce or eliminate growth of microorganisms on the interior surfaces defining the enclosed space.

At (406), the method 400 can include obtaining, by the one or more control devices, data from a measurement device (e.g., optical sensor) while the HINS light sources are activated. The data can be indicative of optical characteristics of light emitted onto the optical sensor. At (408), the data from the measurement device can be processed to determine a wavelength or multiple wavelengths associated with the light emitted onto the measurement device.

At (410), the method 400 can include comparing the wavelengths with a predefined range of HINS wavelengths associated with HINS light. The predefined range of HINS wavelengths can be, for instance, 380 nm to 420 nm, 400 nm to 420 nm, 405 nm, or any other wavelength or range of wavelengths between 380 nm and 420 nm.

When the light emitted onto the measurement device includes light having a wavelength that is within the range of HINS wavelengths, the method 400 can optionally include at (412) providing an indicator signaling that HINS light is being emitted. In the event (410) is not performed when the light is emitted onto the measurement device includes light having a wavelength that is within the range of HINS wavelengths, the method 400 can continue obtaining data from the measurement device as shown in FIG. 6.

When the light emitted onto the measurement device does not include light having a wavelength that is within the range of HINS wavelengths, the method 400 can include at (414) providing an indicator signaling that HINS light is no longer being emitted. In this way, the method 400 can be used to notify a user that HINS light sources are no long illuminating the one or more surfaces defining the enclosed space with HINS light.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A lighting system for an enclosed space associated with a non-occupancy function, the lighting system comprising:
one or more high intensity narrow spectrum (HINS) light sources disposed within the enclosed space, the one or more HINS light sources configured to emit HINS light onto one or more surfaces defining the enclosed space associated with the non-occupancy function.

2. The lighting system of claim 1, further comprising:
one or more control devices configured to control operation of the one or more HINS light sources.

3. The lighting system of claim 2, wherein the one or more control devices are configured to:
obtain data indicative of whether a door movable between an open position and a closed position to permit selective access to the enclosed space is in the open position or the closed position;
activate the one or more HINS light sources when the data indicates the door is in the closed position; and
deactivate the one or more HINS light sources when the data indicates the door is in the open position.

4. The lighting system of claim 2, further comprising:
one or more non-HINS light sources disposed within the enclosed space, the one or more non-HINS light sources configured to emit non-HINS light.

5. The lighting system of claim 4, wherein when a door movable between an open position and a closed position to permit selective access to the enclosed space is in the open position, the one or more control devices are configured to:
control operation of the one or more HINS light sources and the one or more non-HINS light sources to provide a blend of HINS light and non-HINS light.

6. The lighting system of claim 2, further comprising:
an optical sensor configured to generate one or more signals indicative of one or more optical properties of light emitted onto the optical sensor.

7. The lighting system of claim 6, wherein the one or more control devices are configured to:
activate the one or more HINS light sources;
obtain the one or more signals indicative of the one or more optical properties of the light emitted onto the optical sensor; and
deactivate the one or more HINS light sources when the one or more signals indicate HINS light has been emitted onto the optical sensor for a predetermined amount of time.

8. The lighting system of claim 6, wherein the one or more control devices are further configured to:
control operation of one or more output devices external to the enclosed space to provide at least one indicator based, at least in part, on the one or more signals indicative of the one or more optical properties of the light emitted onto the optical sensor.

9. The lighting system of claim 8, wherein the at least one indicator is indicative of the one or more HINS light sources emitting HINS light.

10. The lighting system of claim 8, wherein the at least one indicator is indicative of the one or more HINS light sources not emitting HINS light.

11. The lighting system of claim 8, wherein the at least one indicator comprises at least one of a visual indicator and an audible indicator.

12. The lighting system of claim 1, wherein the HINS light has a wavelength ranging from about 380 nanometers to about 420 nanometers.

13. The lighting system of claim 1, wherein the HINS light has a wavelength of about 405 nanometers.

14. A method of controlling operation of a lighting system for an enclosed space associated with storing inanimate objects, the method comprising:
obtaining, by one or more control devices, data indicative of a trigger event; and
responsive to obtaining the data indicative of the trigger event, activating, by the one or more control devices, one or more high intensity narrow spectrum (HINS) light sources of the lighting system, the one or more HINS light sources disposed within the enclosed space associated with storing inanimate objects such that the one or more or more HINS light sources emit light onto one or more surfaces defining the enclosed space.

15. The method of claim 14, wherein the trigger event is associated with a door movable between an open position and a closed position to permit selective access to the enclosed space being in the closed position.

16. The method of claim 15, further comprising:
deactivating, by the one or more control devices, the one or more HINS light sources when the data indicates the door is no longer in the closed position.

17. The method of claim 14, further comprising:
obtaining, by the one or more control devices, data indicative of one or more optical properties of light the one or more HINS light sources emit onto an optical sensor disposed within the enclosed space associated with storing inanimate objects.

18. The method of claim 17, further comprising:
determining, by the one or more control devices, whether the light emitted onto the optical sensor comprises HINS light based, at least in part, on the data; and
responsive to determining the light includes HINS light, providing, by the one or more control devices, at least one indicator via one or more output devices of the lighting system.

19. A lighting system for an enclosed space associated with an appliance, the lighting system comprising:
one or more high intensity narrow spectrum (HINS) light sources disposed within the enclosed space, the one or more HINS light sources configured to emit HINS light onto one or more surfaces defining the enclosed space associated with the appliance.

20. The lighting system of claim 19, further comprising:
one or more non-HINS light sources disposed within the enclosed space, the one or more non-HINS light sources configured to emit non-HINS light onto the one or more surfaces defining the enclosed space associated with the appliance.
